(19) United States
(12) Patent Application Publication
(10) Pub. No.: US 2017/0312297 A1
(43) Pub. Date: Nov. 2, 2017

(54) Long Acting Pharmaceutical Compositions For Hepatitis C

(71) Applicant: GlaxoSmithKline Intellectual Property (No. 2) Limi, Brentford, Middlesex (GB)

(72) Inventors: Zhi HONG, Research Triangle Park, NC (US); Martin R. LEIVERS, Research Triangle Park, NC (US)

(73) Assignee: Glaxosmithkline Intellectual Property (NO. 2) Limited, Middlesex (GB)

(21) Appl. No.: 15/525,910

(22) PCT Filed: Oct. 30, 2015
(86) PCT No.: PCT/IB2015/058405

§ 371 Date: May 10, 2017

Related U.S. Application Data
(60) Provisional application No. 62/077,647, filed on Nov. 10, 2014.
(60) Provisional application No. 62/077,980, filed on Nov. 11, 2014.
(60) Provisional application No. 62/092,499, filed on Dec. 16, 2014.

(51) Int. Cl. A61K 31/69; A61K 9/00; A61K 47/10; A61K 31/7072; A61K 31/7068; A61K 31/549; A61K 31/519; A61K 31/506; A61K 31/4709; A61K 31/439; A61K 31/4188; A61K 31/4184; A61K 31/4178; A61K 9/50; A61K 47/12; A61K 47/26

(52) U.S. Cl. CPC A61K 31/69; A61K 47/26; A61K 9/0019; A61K 47/10; A61K 31/7072; A61K 31/7068; A61K 31/549; A61K 31/519; A61K 31/506; A61K 31/4709; A61K 31/439; A61K 31/4188; A61K 31/4184; A61K 31/4178; A61K 9/5031; A61K 47/12

(57) ABSTRACT

The present Invention relates to long acting pharmaceutical compositions useful in the treatment or prevention or cure of viral infections, such as HCV infections, and diseases associated with such infections.

### CROSS-REFERENCE TO RELATED PATENTS AND PATENT APPLICATIONS

This is a Patent Cooperation Treaty application and claims the benefit of U.S. Provisional Application Ser. No. 62/077,647, filed Nov. 10, 2014; U.S. Provisional Application Ser. No. 62/077,980; filed Nov. 11, 2014; and U.S. Provisional Application Ser. No. 62/092,499; filed Dec. 16, 2014.

### FIELD OF THE INVENTION

The present invention relates to long acting parenteral (“LAP”) formulations of anti-viral agents, specifically Hepatitis C Virus (HCV) active compounds as well as methods of treating or preventing or curing viral infections, such as HCV infections, and diseases associated with such infections.

### BACKGROUND OF THE INVENTION

Infection with HCV is a major cause of human liver disease throughout the world. Chronic infection with HCV is associated with chronic liver disease, cirrhosis, hepatocellular carcinoma, and liver failure. HCV is a hepacivirus member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-N52-N53-NS4a-NS4b-NS5a-NS5b. While the pathology of HCV infection affects mainly the liver, the virus is found in other cell types in the body including peripheral blood lymphocytes.

HCV is a major causative agent for post-transfusion and for sporadic hepatitis. Infection by HCV is insidious in a high proportion of chronically infected, and infectious, carriers who may not experience clinical symptoms for many years. An estimated 170 million chronic carriers worldwide are at risk of developing liver disease.

Due to the high degree of variability in the viral surface antigens, existence of multiple viral genotypes, and demonstrated specificity of immunity, the development of a successful vaccine in the near future is unlikely. Alpha-interferon, alone or in combination with ribavirin, has been widely used for treatment of chronic HCV infection. However, treatment of HCV with interferon has frequently been associated with adverse side effects such as fatigue, fever, chills, headache, leukopenia, thrombocytopenia, psychiatric effects and associated disorders, autoimmune phenomena and associated disorder and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current therapy of interferon-alpha (IFN) and ribavirin. With the introduction of pegylated interferon, both initial and sustained response rates have improved, and combination treatment of Peg-IFN with ribavirin until recently, constituted a standard for therapy. However, the side effects associated with combination therapy persist. Ribavirin causes significant hemolysis in 10-20% of patients treated at currently recommended doses, and the drug is both teratogenic and embryotoxic.

Most recently, oral agents including Sofosbuvir were introduced as a component of a combination antiviral regimen for patients with HCV mono-infection and HCV/HIV-1 coinfection. Treatment regimen and duration are dependent on both viral genotype and patient population and can vary from 8 to 24 weeks. Consequently, a prescribed treatment requires ingestion of a daily regimen which can lead to reduced patient compliance resulting in reduced drug efficacy and development of resistant strains of HCV. In highly motivated populations, adherence to these shorter duration therapies can be good and cure rates can be very high. In marginal populations such as IV drug abusers, the homeless, and the mentally ill, adherence to regimens may be poorer and a lack of adherence may result in treatment failure and development of long-lived resistance mutations in the HCV genome. Additionally for some populations, such as incarcerated patients, the associated cost of each treatment (dose) may be very high.

Accordingly, successful long acting treatments for HCV infected patients which reduce the number of treatments down to even a single treatment can alleviate compliance issues and issues associated with the cost of treatment This would represent a significant advance for HCV patients.

PCT Published Application No. WO2013028371 deriving from U.S. Provisional Application 61/525,440, filed Aug. 19, 2011, discloses benzofuran derivatives for the treatment of Hepatitis C Virus (HCV). Such benzofuran derivatives include 6-(N-(7-chloro-1-hydroxy-1,3-dihydrobenzo[c][1,2]oxaborol-5-yl)methylsulfonamido)-5-cyclopropyl-2-(4-fluorophenyl)-N-methylbenzofuran-3-carboxamide which is the compound of Formula I,

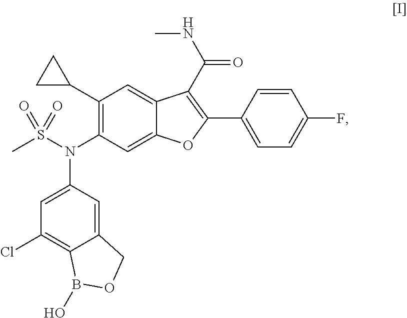 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof.

### SUMMARY OF THE INVENTION

The present invention addresses potential issues of non-compliance and increased patient convenience as well as treatment or cure or prevention of strains of HCV by formulating certain benzofuran derivatives, including the compound of Formula I, as LAP compositions suitable for administration, for example, once, once per month, once every 2 months, once every 3 months, once every 6 months, or once every 12 months.

In a first aspect of the present invention, there is provided a LAP pharmaceutical composition including at least one benzofuran derivative or a pharmaceutically acceptable salt thereof.

In a second aspect of the present invention, there is provided a LAP pharmaceutical composition including the compound of Formula I

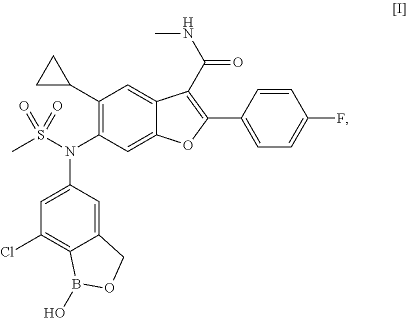 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof.

In a third aspect of the present invention, there is provided a method for the treatment of an HCV infection in a human having an HCV infection including administering to the human a LAP pharmaceutical composition including at least one benzofuran derivative or a pharmaceutically acceptable salt thereof.

In a fourth aspect of the present invention, there is provided a method for the treatment of an HCV infection in a human having an HCV infection including administering to the human a LAP pharmaceutical composition including the compound of Formula I

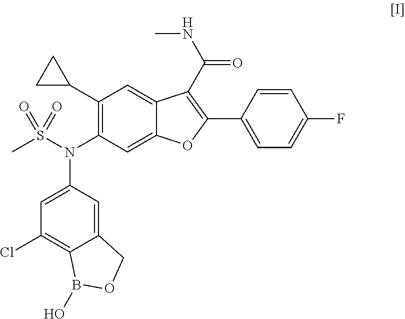 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof.

In a fifth aspect of the present invention, there is provided use of a LAP pharmaceutical composition including at least one benzofuran derivative or a pharmaceutically acceptable salt thereof in medical therapy.

In a sixth aspect of the present invention, there is provided use of a LAP pharmaceutical composition including the compound of Formula I

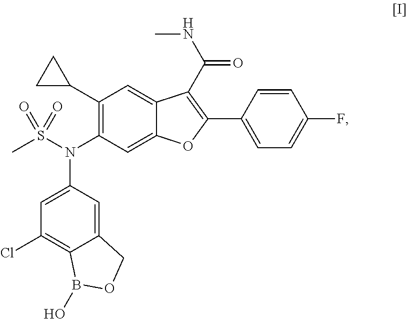 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof in medical therapy.

In a seventh aspect of the present invention, there is provided the use of at least one benzofuran derivative or a pharmaceutically acceptable salt thereof in the preparation of a long acting parenteral medicament for use in the treatment of HCV infection in a human.

In an eighth aspect of the present invention, there is provided the use of a compound of Formula I

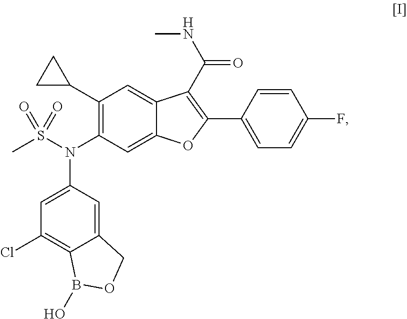 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, in the preparation of a long acting parenteral medicament for use in the treatment of HCV infection in a human.

In a ninth aspect of the present invention, there is provided the use of a compound of Formula I

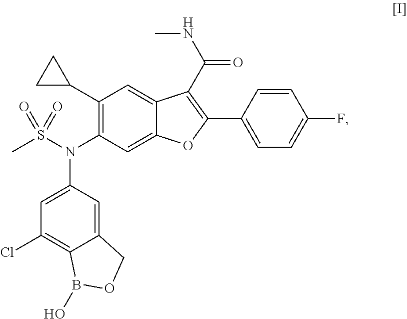 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, in the preparation of a long acting parenteral medicament for use in curing an HCV infection in a human.

In a tenth aspect of the present invention, there is provided the use of a compound of Formula I

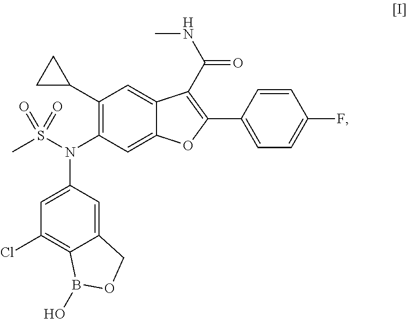 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, in the preparation of a long acting intramuscular parenteral for use as a 1-2 treatment cure of an HCV infection in a human, and in some embodiments, a single treatment cure.

### BRIEF DESCRIPTION OF THE DRAWINGS

FIG. 1 depicts a plot of mean blood concentration of two LAP formulations of the compound of Formula I versus time in hours in rat (intramuscular—IM and subcutaneous—SC).

FIG. 2 depicts a plot of individual blood concentrations of a micronised Poloxamer 188 LAP formulation of the compound of Formula I at 100 mg/kg versus time in hours in dog (intramuscular—IM).

FIG. 3 depicts a plot of individual blood concentrations of a nanosized Poloxamer 188 LAP formulation of the compound of Formula I at 100 mg/kg versus time in hours in dog (intramuscular—IM).

FIG. 4 depicts a plot of individual blood concentrations of a micronized Tween 20 LAP formulation of the compound of Formula I at 10 mg/kg versus time in hours in dog (intramuscular—IM).

FIG. 5 depicts a plot of individual blood concentrations of a nanosized Tween 80 LAP formulation of the compound of Formula I at 10 mg/kg versus time in hours in dog (intramuscular—IM).

### DETAILED DESCRIPTION OF THE INVENTION

Hepatitis C virus is a positive strand RNA virus. The key enzyme for HCV RNA synthesis is NS5B, the RNA-dependent RNA polymerase that replicates the viral genome. NS5B works in a membrane-associated complex that also contains NS4A, NS4B, NS3 protease-helicase and NSSA. These subunits can recognize cis-acting regulatory sequences in the HCV genome. These proteins also have some additional roles during the infection process that are independent of RNA synthesis. Therefore, targeting the viral replication enzymes could prevent the virus from affecting normal cellular processes as well as inhibiting HCV RNA synthesis.

Harvoni® is a recently approved combination of the NS5B polymerase inhibitor Sofosbuvir coformulated with the NSSA inhibitor ledipisvir for the treatment of HCV genotypes 1. Phase 3 trials of Harvoni® involving patients with HCV alone have demonstrated it to be effective when used for 8-24 weeks for HCV genotype 1. Other combinations of oral agents such as Sofosbuvir and ribavirin have been shown to be effective in treating other genotypes of HCV. Although there are effective treatment regimens, they all require daily ingestion which can lead to reduced patient compliance resulting in reduced drug efficacy and resistance.

6-(N-(7-chloro-1-hydroxy-1,3-dihydrobenzo[c][1,2]oxaborol-5-yl)methylsulfonamido)-5-cyclopropyl-2-(4-fluorophenyl)-N-methylbenzofuran-3-carboxamide which is the compound of Formula I,

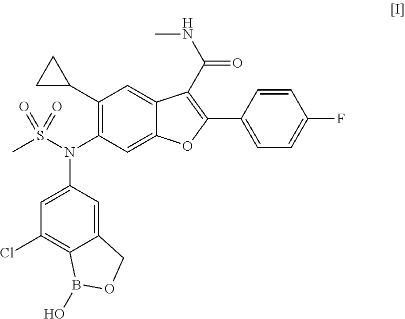 [SMILES: CNC(=O)c1c(-c2ccc(F)cc2)oc2cc(N(c3cc(Cl)c4c(c3)COB4O)S(C)(=O)=O)c(C3CC3)cc12]

is an NS5B polymerase inhibitor that is currently being developed for the treatment or prevention or cure of HCV infections and associated disease states.

The present invention addresses ease of treatment and non-compliance issues in the treatment of HCV by formulating a benzofuran derivative, including 6-(N-(7-chloro-1-hydroxy-1,3-dihydrobenzo[c][1,2]oxaborol-5-yl)methylsulfonamido)-5-cyclopropyl-2-(4-fluorophenyl)-N-methylbenzofuran-3-carboxamide (the compound of Formula I) as a long-acting parenteral (LAP) composition or depot formulation suitable for administration, for example, once, once per week, once every two weeks, once per month, once every 2 months, once every 3 months, once every 6 months or once every 12 months.

Long-acting parenteral formulations of “benzofuran derivatives” (e.g., the compound of Formula I) could generate sustained effective inhibitory concentrations with infrequent dosing and may improve adherence to therapy. Next to facilitating maintenance of viral suppression following traditional anti-HCV therapy, a long-acting formulation, may also serve as a practical opportunity for pre-exposure prophylaxis.

The present invention features pharmaceutical compositions comprising an active ingredient which is the compound of Formula I, or a pharmaceutically acceptable salt thereof, suitable for administration once, once monthly or longer.

The present invention is expected to result in prolonged plasma exposure of compound of Formula I at concentrations above that minimally required for suppression of the HCV virus from a single treatment. With prolonged suppression of the virus, normally longer than 6 weeks, a sustained virologic response can be achieved resulting in functional cure of HCV. The single treatment may be comprised of single or multiple injections (eg 1, 2, 3 or 4 injections) given within a short period of time, say less than one hour. Reducing the treatment phase to a single day results in significant advantages including greater compliance with a full curative regimen, reduced healthcare utilization and allowance of a test and treat paradigm.

Further features of the present invention are methods of using these pharmaceutical compositions.

In one embodiment, the present invention features pharmaceutical compositions, comprising a compound of Formula I, or a pharmaceutically acceptable salt thereof, and a surfactant system.

Pharmaceutically acceptable salts include, but are not limited to those described in PCT Published Application No. WO2013028371 deriving from U.S. Provisional Application 61/525,440, filed Aug. 19, 2011.

The term “therapeutically effective amount,” as used herein, means a sufficient amount of a drug, compound, composition, product or pharmaceutical agent to abate or reverse or treat a malady in a human or other mammal.

The present invention features parenteral pharmaceutical compositions for administration to a subject, for example a human.

In another embodiment, the present invention features long-acting parenteral pharmaceutical compositions comprising a compound of formula (I) or a pharmaceutically acceptable salt thereof, and a surfactant system for a single administration which may be in the form of one to three injections.

In another embodiment, the present invention features long-acting parenteral pharmaceutical compositions comprising a compound of formula (I) or a pharmaceutically acceptable salt thereof, and a surfactant system for weekly (once every week) administration.

In another embodiment, the present invention features long-acting parenteral pharmaceutical compositions comprising a compound of formula (I) or a pharmaceutically acceptable salt thereof, and a surfactant system for bi-weekly (once every two weeks) administration.

In another embodiment, the present invention features long-acting parenteral pharmaceutical compositions comprising a compound of formula (I) or a pharmaceutically acceptable salt thereof, and a surfactant system for once monthly administration.

In another embodiment, the present invention features long-acting parenteral pharmaceutical compositions comprising a compound of formula (I) or a pharmaceutically acceptable salt thereof, and a surfactant system for bi-monthly (once every two months) administration.

In another embodiment, the present invention features long-acting parenteral pharmaceutical compositions comprising a compound of formula (I) or a pharmaceutically acceptable salt thereof, and a surfactant system for tri-monthly (once every three months) administration.

In another embodiment, the present invention features long-acting parenteral pharmaceutical compositions comprising a compound of formula (I) or a pharmaceutically acceptable salt thereof, and a surfactant system administration once every six or twelve months, or any time point within this range.

The compositions of the present invention provide for the slow release of a compound of formula (I) over an extended period of time within the body of a subject. Therefore, in order to achieve therapeutic levels of drug, a compound of formula (I) advantageously is released from the composition within approximately one to three months, or any time point within this range.

An embodiment of the present invention is a pharmaceutical composition suitable for parenteral administration comprising a compound of formula (I) and a surfactant system comprising a combination of polymers providing for the release of a compound of formula (I) over a period of one week to three months. A suitable combination of polymers is, for example, polysorbate 80 and polyvinylpyrrolidone (PVP).

The compositions of the present invention may be administered to the subject by various routes, including intramuscular (IM), intravenous (IV), or subcutaneous (SC). Therefore, in one embodiment, the compositions of the present invention are administered to a subject by an intramuscular route. In another embodiment, the compositions of the present invention are administered to a subject by an intravenous route. In another embodiment, the compositions of the present invention are administered to a subject by a subcutaneous route.

For purposes of the present invention, a “surfactant system” means any formulation suitable for pharmaceutical purposes that includes at least one surfactant. For example, a surfactant system that can be used with the present invention may include, in addition to a surfactant, additional components such as buffers, polymers (for drug particles), wetting agents, stabilizers, tonicity modifiers, and solvents such as water.

The surfactant system may include any surfactant as long as it is compatible with pharmaceutical applications. For example, suitable surfactants include, but are not limited to, polyoxyethylene sorbitan fatty acid esters (polysorbates such as polysorbate 20 or 80), poloxamers (such as LUTROL™ F68, F108 and F127 which are block copolymers of ethylene oxide and propylene oxide, sodium dodecylsulfate and/or sodium lauryl sulphate), sorbitan esters of fatty acids (SPAN), polyethoxylated castor oil and its derivatives, tocopheryl polyethylene glycol succinate, and polyvinyl alcohols. In certain embodiments, the surfactant system comprises an amount of surfactant that ranges from about 0.01% (w/v) to about 5% (w/v) surfactant. In other embodiments, the surfactant system comprises an amount of surfactant that ranges from about 0.1% (w/v) to about 3% (w/v) surfactant. In still other embodiments, the surfactant system comprises about 0.2% (w/v) surfactant. In still other embodiments, the surfactant system comprises about 0.4% (w/v) surfactant. In other embodiments, the surfactant system comprises polysorbate-80 (e.g., Tween-80). In still other embodiments, the surfactant system comprises 0.4% (w/v) polysorbate-80.

Representative stabilizers include, but are not limited to, polyethylene glycols, carboxymethylcellulose calcium, carboxymethylcellulose sodium, methylcellulose, hydroxyethylcellulose, hydroxypropylcellulose, hydroxymethylpropylcellulose, polysaccharides, hyarluronic acid, polyvinyl alcohol (PVA) and polyvinylpyrrolidone (PVP). In certain embodiments, the surfactant system comprises an amount of stabilizer that ranges from about 0.01% (w/v) to about 5% (w/v) stabilizer. In other embodiments, the surfactant system comprises an amount of stabilizer that ranges from about 1% (w/v) to about 5% (w/v) stabilizer. In other embodiments, the surfactant system comprises an amount of stabilizer that ranges from about 1% (w/v) to about 3% (w/v) stabilizer. In still other embodiments, the surfactant system comprises about 2% (w/v) stabilizer. In other embodiments, the surfactant system comprises polyethylene glycols. In other embodiments, the surfactant system comprises PEG-3350. In still other embodiments, the surfactant system comprises 2% (w/v) PEG-3350.

Suitable buffer salts include, but are not limited to, buffer salts selected from phosphate salts, citrate salts, acetate salts, and tartrate salts, etc. In certain embodiments, the surfactant system comprises an amount of buffer salts that ranges from about 1 mM to about 100 mM buffer salt. In other embodiments, the surfactant system comprises an amount of buffer salts that ranges from about 2 mM to about 50 mM buffer salt. In other embodiments, the surfactant system comprises an amount of buffer salts that ranges from about 3 mM to about 25 mM buffer salt. In other embodiments, the surfactant system comprises an amount of buffer salts that ranges from about 5 mM to about 15 mM buffer salt. In still other embodiments, the surfactant system comprises about 10 mM buffer salt. In certain embodiments, the pH of the buffer salt is adjusted to range from about pH 6.0 to about pH 8.0. In other embodiments, the pH of the buffer salt is adjusted to range from about pH 6.5 to about pH 7.5. In other embodiments, the pH of the buffer salt is adjusted to range from about pH 6.7 to about pH 7.3. In one embodiment, the buffer salt comprises phosphate buffered saline (PBS). In another embodiment, the buffer salt comprises phosphate buffered saline at a concentration of about 10 mM. In another embodiment, the buffer salt comprises phosphate buffered saline at a concentration of about 10 mM and a pH of about 6.9.

Suitable tonicity modifiers include, but are not limited to, sodium chloride, mannitol, sucrose, maltose, and dextrose, etc. In one embodiment, the tonicity modifier comprises sodium chloride. In another embodiment, the tonicity modifier is sodium chloride. In certain embodiments, the surfactant system comprises a concentration of tonicity modifier that ranges from about 0 to about 350 mM. In certain embodiments, the surfactant system comprises a concentration of tonicity modifier that ranges from about 0 to about 175 mM. In certain embodiments, the surfactant system has a tonicity that ranges from about 250 to about 350 mOsmol/kg.

In one embodiment, the compound of Formula I can be suspended as microparticles in a surfactant system and aqueous buffer. In some embodiments, the compound of Formula I can be in an amorphous form or in a crystalline form. Typically, the drug particle size (D₅₀) will range from about 0.05 μm to about 100 μm. In other embodiments, the drug particle size will range from about 0.1 μm to about 50 μm. In other embodiments, the drug particle size will range from about 0.1 μm to about 20 μm. In other embodiments, the drug particle size (D₅₀) will range from about 0.1 μm to about 10 μm. In other embodiments, the drug particle size (D₅₀) will range from about 0.1 μm to about 5 μm. In other embodiments, the drug particle size (D₅₀) will range from about 1 μm to about 5 μm. In other embodiments, the drug particle size (D₅₀) will range from about 0.05 μm to about 0.05 μm. In other embodiments, the drug particle size (D₅₀) will range from about 0.5 μm to about 5 μm. In other embodiments, the drug particle size (D₅₀) will range from about 5 μm to about 25 μm. In other embodiments, the drug particle size (D₅₀) will range from about 25 μm to about 100 μm.

In still other embodiments, the drug particle size in the surfactant system can be mixed sizes. For example, having substantially different particle sizes from relatively large to relatively small, can achieve acceptable pharmacokinetic parameters for the formulation because the small particles are absorbed and metabolized quicker than the larger particles. This type of mixed particle size formulation could enhance the long acting nature of the present invention by providing a quicker release of drug to the subject early after administration while still maintaining a long acting release of the drug at distant times after administration. Therefore, in one embodiment, the present LAP invention could comprise two or more substantially different particle sizes that would allow for earlier and later release of the compound of Formula I and such differing absorption kinetics would be a means of enhancing a durable long acting drug exposure. In one embodiment, the compound of Formula I is in a microparticle form, wherein the microparticles of the compound of Formula I range in size from about 0.05 μm to about 100 μm, wherein said microparticles comprise two or more substantially different particle sizes.

In still other embodiments, the drug particles of the compound of Formula I are encapsulated into polymer based microparticles that can, optionally, be subsequently freeze dried for extended storage. When the term “encapsulated” is used with regards to the present invention, it is meant that the compound of Formula I is substantially surrounded by a polymer even though some compound may still be present on the surface of the encapsulated compound/polymer structure. Immediately before use, the dry microparticles can optionally be suspended in an aqueous buffer solution. The polymers used to prepare such microparticles can be selected from a series of biodegradable polymers including poly (lactic-co-glycolic) acid (M_{w} 5-200 kD) and its derivatives, such as polyethylene glycol based amphiphilic polymers, etc. The microparticle size (D₅₀) could range from about 1 μm to about 100 μm and the drug encapsulation could range from about 10% to about 70% (w/w). In one embodiment, the drug particles of the compound of Formula I are encapsulated into polymer based microparticles such as those containing Resomer™. In another embodiment, the drug particles of the compound of Formula I are encapsulated into polymer based microparticles such as those containing Resomer™ 752S.

In other embodiments, in-situ gels could be used to encapsulate the compound of Formula I. This could be a water-miscible organic solvent-based solution that contains both the compound of Formula I and a gel-forming polymer that is water-insoluble. Once administrated (IM or SC), the organic solvent dissipates away and the water-insoluble polymer precipitates out to form the gel containing the compound of Formula I. The compound of Formula I would then slowly diffuse out as the polymer-based gel degrades in body. The polymers used to prepare in-situ gels are selected from a series biodegradable polymers including poly (lactic-co-glycolic) acid (M_{w} 5-200 kD) and its derivatives, polyethylene glycol based amphiphilic polymers, etc. The organic solvents are selected from N-methyl pyrrolidone (NMP), dimethylsulfoxide (DMSO), dimethylformamide (DMF), dimethylacetamie (DMA), etc. The concentration of the polymer in the organic solvent could be between 1-50% (w/w) and the compound of Formula I concentration could be between 1-50% (w/w).

Alternatively, the microparticle formulation can be made through spray-drying process. Similarly, the organic solution containing both the compound of Formula I and the selected polymer prepared as described herein is subjected to a spray-drying process where the organic solvent is rapidly evaporated under nitrogen gas flow to form the compound of Formula I encapsulated microparticles. The drying temperature is no less than 35 C and the solution spray rate is no less than 0.1 ml/min. For the in-situ gel microparticles, the compound of Formula I and the selected polymer could be co-dissolved into the suitable organic solvent wherein the organic solvent must meet the following criteria: a) has a good solubility for the selected polymer; b) has a good miscibility with aqueous solution; and c) has a low toxicity and demonstrated safety when use in human; for example N-methyl pyrrolidone (NMP), dimethylsulfoxide (DMSO), dimethylformamide (DMF), dimethylacetamie (DMA), etc. The resulted solution containing both the compound of Formula I and selected polymer can be formulated by varying the polymer concentration, the polymer to the compound of Formula I ratio in the solvent so as to control the gel forming rate after administration and the subsequent drug diffusion rate. The solution finally is subjected to a terminal sterilization by γ-irradiation on dry ice at a minimum dose of 25 kGy.

An example of a combination of polymers includes a polysorbate, for example, polysorbate 80 as wetting agent and a polyvinylpyrrolidone (PVP), for example, Plasdone K29/32 as a stabilizer. Therefore, in one embodiment, the present invention features a parenteral pharmaceutical composition comprising a compound of formula (I), or a pharmaceutically acceptable salt thereof, and polysorbate 80 and the polyvinylpyrrolidone: Plasdone K29/32.

An embodiment of the present invention is a pharmaceutical composition for parenteral administration comprising a compound of formula (I) and a surfactant system suitable for commonly known sterilization technologies such as gamma irradiation, electron beam irradiation and autoclave sterilization.

An embodiment of the present invention is a pharmaceutical composition for parenteral administration comprising a compound of formula (I) and a surfactant system that can be manufactured using aseptic technique.

An embodiment of the present invention is a pharmaceutical composition for parenteral administration comprising a compound of formula (I) and a surfactant system suitable for gamma radiation sterilization.

An embodiment of the present invention is a pharmaceutical composition for parenteral administration comprising a compound of formula (I) and a surfactant system suitable for sterilization technologies by electron beam irradiation or autoclave sterilization.

An embodiment of the present invention is a pharmaceutical composition for parenteral administration that can be presented as a “ready to use” sterile suspension or lyophile for reconstitution.

The compositions of the present invention may be administered by subcutaneous or intramuscular injection. The compositions of the present invention may also be administered by intradermal or intravitreal injection or implant. The compositions of the present invention may also be administered by other parenteral routes of administration.

The preparation of the compositions of the present invention may be performed by milling using a wet bead mill and sterilized by gamma irradiation.

Another feature of the present invention is to simplify treatment regimens and provide cure regimens for HCV with the goal of enhancing patient compliance by providing a simplified dosage form containing therapeutically effective amounts of a compound of formula (I) or a pharmaceutically acceptable salt thereof.

The present invention also features a method for treating or curing HCV infections in a human, which method comprises administering to said human a composition according to the invention. The present invention features the use of a pharmaceutical composition according to the invention in the treatment or cure of HCV infections. The present invention features the manufacture of a medicament according to the invention for use in medical therapy. The present invention features the manufacture of a medicament according to the invention for use in the treatment or cure of HCV infection.

The present invention also features a method for treating or curing HCV infections in a human which method comprises administering to said human a composition according to the invention before, during, or after therapy with a compound of formula (I) in tablet or solution form.

It will be appreciated by those skilled in the art that reference herein to “treatment” or “treating” or “treat” extends to the treatment of an established malady, infection or symptoms thereof. It will also be appreciated by those skilled in the art that reference herein to “cure” or “curing” extends to a patient having a complete recovery from an established malady, infection or symptoms thereof.

The present invention also features a method for preventing HCV infections in a human, which method comprises administering to said human a composition according to the invention. The present invention features the use of a pharmaceutical composition according to the invention in the prevention of HCV infections. The present invention features the manufacture of a medicament according to the invention for use in prophylactic medical therapy. The present invention features the manufacture of a medicament according to the invention for use in preventing HCV infection.

The present invention also features a method for treating or preventing HCV infections in a human which method comprises administering to said human a composition according to the invention before, during, or after therapy with a compound of formula (I) in tablet or solution form.

Therefore, in certain embodiments of the present invention, there is provided a single treatment pharmaceutical composition comprising a therapeutically effective amount of a long acting formulation comprising a compound of formula (I):

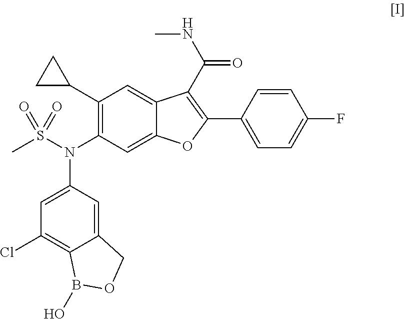 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, in a pharmaceutically acceptable carrier for parenteral administration.

In other embodiments, there is provided a parenteral pharmaceutical composition comprising a compound of formula (I):

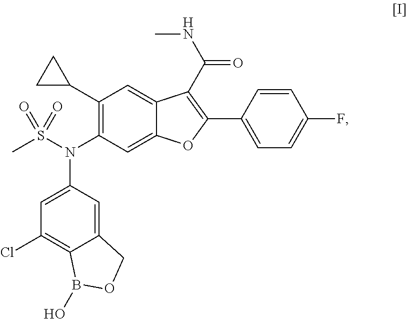 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof.

In other embodiments, there is provided a pharmaceutical composition comprising a compound of formula (I) that is formulated for subcutaneous administration.

In other embodiments, there is provided a pharmaceutical composition comprising a compound of formula (I) that is formulated for intramuscular administration.

In other embodiments, there is provided a pharmaceutical composition comprising a compound of formula (I) that is formulated for administration once.

In other embodiments, there is provided a pharmaceutical composition comprising a compound of formula (I) that is formulated for administration once weekly or longer.

In other embodiments, there is provided a pharmaceutical composition comprising a compound of formula (I) that is formulated for administration once weekly.

In other embodiments, there is provided a pharmaceutical composition comprising a compound of formula (I) that is formulated for administration once per month.

In other embodiments, there is provided a pharmaceutical composition comprising a compound of formula (I) that is formulated for administration once every two months. In other embodiments, there is provided a pharmaceutical composition comprising a compound of formula (I) that is formulated for administration once every three months. In other embodiments, there is provided a pharmaceutical composition comprising a compound of formula (I) that is formulated for administration at any interval between 30 and 365 days.

In other embodiments, there is provided a pharmaceutical composition comprising a compound of formula (I), wherein the compound of formula (I) is present in the composition in the form of crystalline nanoparticles.

In other embodiments, there is provided a pharmaceutical composition comprising a compound of formula (I), wherein the compound of formula (I) is present in the composition in the form of matrix release particles.

In other embodiments, there is provided a pharmaceutical composition comprising a compound of formula (I), wherein the composition can be terminally sterilized by gamma irradiation.

In other embodiments, there is provided a method for the treatment of an HCV infection in a human having an HCV infection comprising administering to the human a single treatment pharmaceutical composition comprising a therapeutically effective amount of a long acting formulation comprising a compound of formula (I):

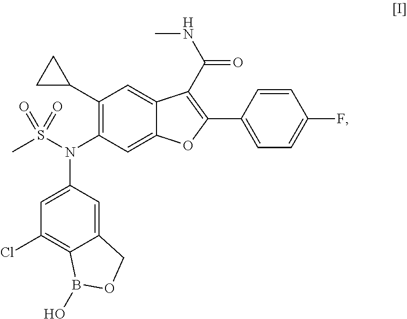 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, in a pharmaceutically acceptable carrier for parenteral administration.

In other embodiments, there is provided a method for the prevention of an HCV infection in a human comprising administering to a human at risk of acquiring an HCV infection, a single treatment pharmaceutical composition comprising a therapeutically effective amount of a long acting formulation comprising a compound of formula (I):

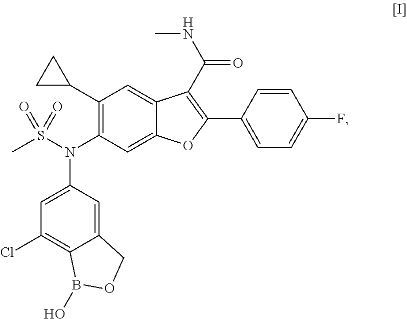 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, in a pharmaceutically acceptable carrier for parenteral administration.

In other embodiments, there is provided a LAP pharmaceutical composition, comprising: at least one benzofuran derivative or a pharmaceutically acceptable salt thereof.

In other embodiments, there is provided a LAP pharmaceutical composition, comprising: the compound of Formula I

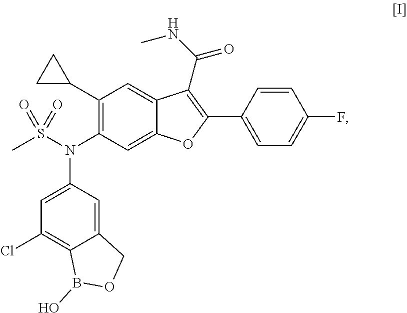 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof.

In other embodiments, there is provided a method for the treatment of an HCV infection in a human having an HCV infection, comprising: administering to the human a LAP pharmaceutical composition including at least one benzofuran derivative or a pharmaceutically acceptable salt thereof.

In other embodiments, there is provided a method for the treatment of an HCV infection in a human having an HCV infection, comprising: administering to the human a LAP pharmaceutical composition including the compound of Formula I

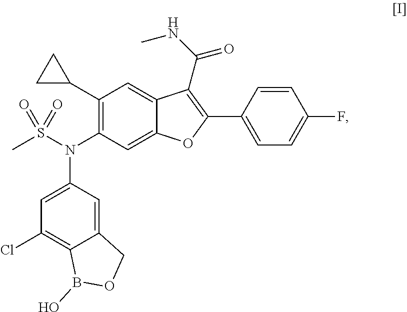 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof.

In other embodiments, there is provided a method for the prevention of a HCV infection in a human having an HCV infection, comprising: administering to the human a LAP pharmaceutical composition including at least one benzofuran derivative or a pharmaceutically acceptable salt thereof.

In other embodiments, there is provided a method for the prevention of an HCV infection in a human having an HCV infection, comprising: administering to the human a LAP pharmaceutical composition including the compound of Formula I

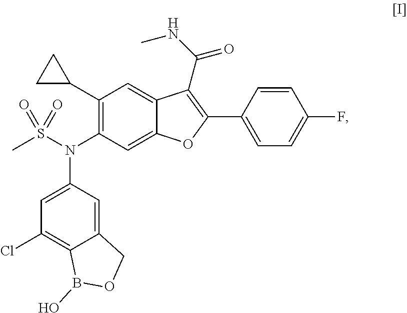 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof.

In other embodiments, there is provided a long acting parenteral (LAP) pharmaceutical composition comprising a compound of Formula I:

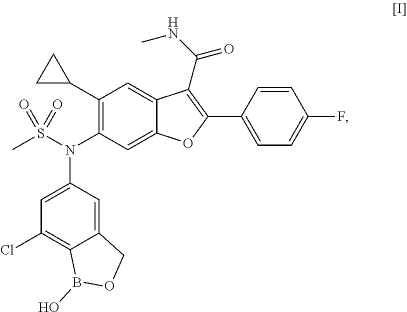 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, and one or more pharmaceutically acceptable excipients that comprise:

a) a surfactant;

b) a stabilizer;

c) a tonicity agent;

d) a buffer; and

e) a solvent.

In other embodiments, there is provided a LAP pharmaceutical composition according, wherein the surfactant above is Tween 20 or Tween 80.

In other embodiments, there is provided a LAP pharmaceutical composition, wherein the buffer above is an acetate buffer.

In other embodiments, there is provided a LAP pharmaceutical composition, wherein the tonicity agent above is mannitol.

In other embodiments, there is provided a LAP pharmaceutical composition above, wherein the tonicity agent is D-Mannitol.

In other embodiments, there is provided a long acting parenteral (LAP) pharmaceutical composition comprising a compound of Formula I:

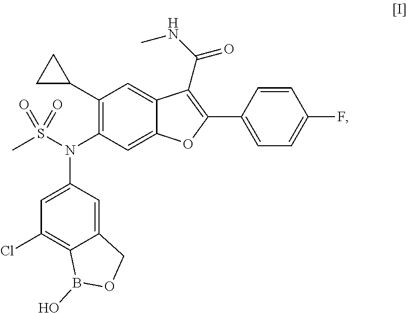 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, and one or more pharmaceutically acceptable excipients that comprise:

a) Poloxamer 188;

b) PEG3350;

c) D-mannitol;

d) a buffer comprising sodium acetate or sodium phosphate or both; and

e) water.

In other embodiments, there is provided a long acting parenteral (LAP) pharmaceutical composition comprising a compound of Formula I:

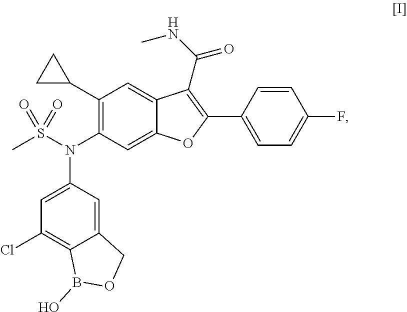 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, wherein the compound of Formula I is present at a concentration that ranges from 100-150 mg/ml, and one or more pharmaceutically acceptable excipients that comprise:

| Component | Function | Concentration (mg/ml) | |
|---|---|---|---|
| Poloxamer 188 | Wetting agent | 50 | |
| PEG3350 | Stabilizer | 20 | |
| Mannitol | Tonicity agent | 45 | |
| Sodium acetate or sodium | Buffer | 20 | mM |
| phosphate | | | |

| | | |
|---|---|---|
| Water | Solvent | Q.S. |

In other embodiments, there is provided a method for curing an HCV infection in a human having an HCV infection, comprising: administering to the human the above LAP pharmaceutical composition.

In other embodiments, there is provided a method of curing an HCV infection in a human comprising administering to the human any of the above LAP pharmaceutical compositions comprising the compound of Formula I, wherein the administration comprises 1-2 injections of the LAP pharmaceutical composition.

In other embodiments, there is provided the method above wherein the administration comprises 1 intramuscular injection of the LAP pharmaceutical composition.

In other embodiments, there is provided a kit comprising a stoppered glass vial comprising a long acting parenteral (LAP) pharmaceutical composition comprising a compound of Formula I:

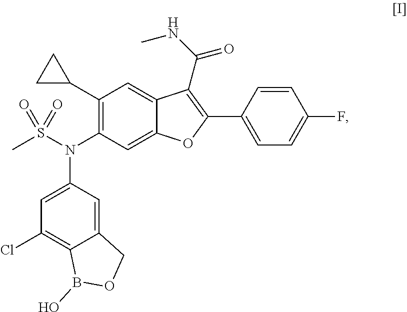 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, and one or more pharmaceutically acceptable excipients that comprise:

a) Poloxamer 188;

b) PEG3350;

c) D-mannitol;

d) a buffer comprising sodium acetate or sodium phosphate or both; and

e) water.

In other embodiments, there is provided a LAP pharmaceutical composition, comprising: the compound of Formula I

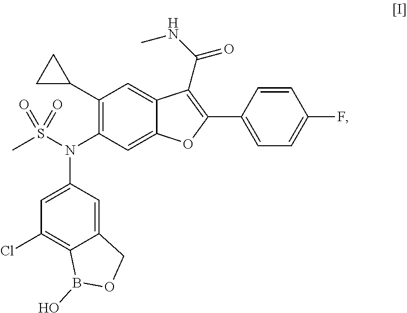 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, further comprising a surfactant system.

In other embodiments, there is provided a LAP pharmaceutical composition, comprising: the compound of Formula I

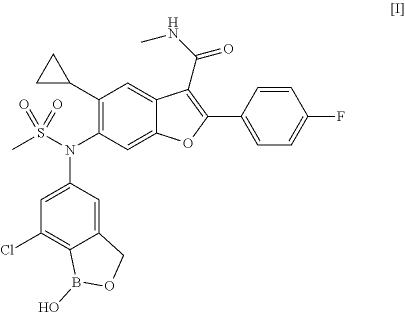 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof, further comprising a surfactant system, wherein the surfactant system comprises a surfactant in an amount ranging from about 0.1% (w/v) to about 3% (w/v) surfactant, or an amount ranging from 0.2% (w/v) to about 0.4% (w/v) surfactant, or the surfactant system comprises about 0.4% (w/v) surfactant.

In other embodiments, there is provided a LAP pharmaceutical composition, comprising: the compound of Formula I

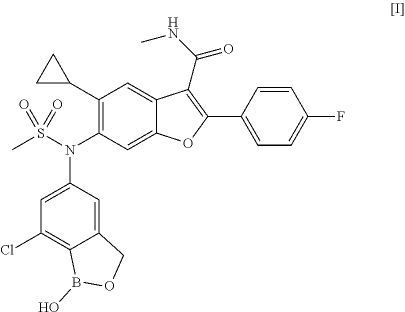 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof,

in combination with one or more additional compounds selected from the group consisting of Telaprevir (Incivek®), Boceprevir (Victrelis®), ABT-450, Faldaprevir (BI-201335), Asunaprevir (BMS-650032), GS-9256, GS-9857, ABT-493, Vedroprevir (GS-9451), Danoprevir (ITMN-191, RG7227), (Grazoprevir) MK-5172, Vaniprevir (MK-7009), Sovaprevir (ACH-1625), Deldeprevir (Neceprevir) (ACH-2684), Narlaprevir (SCH 900518), Simeprevir (TMC 435), ABT-267, ABT-530, Daclatasvir, Velpatasvir, Ledipasvir, ACH-2928, odalasvir (ACH-3102), PPI-668, AZD-7295, Elbasvir (MK-8742), MK-8408, BMS-986094, MK-3862 (IDX-21437), Sofosbuvir, AL-335, GS-0938, Mericitabine, BCX-5191, IDX-184, ALS-2200 (VX-135), ALS-2158, TMC649128, VX-222, ABT-072, ABT-333, Deleobuvir (BI-207127), Tegobuvir (GS-9190), Setrobuvir (ANA-598), CC-31244, Filibuvir (PF-868554), VCH-916, VCH-759, BMS-791325, TMC-647055, TKM-HCV, or a pharmaceutically salt thereof.

In other embodiments, there is provided a method for the treatment of an HCV infection in a human having an HCV infection, comprising: administering to the human a LAP pharmaceutical composition including the compound of Formula I

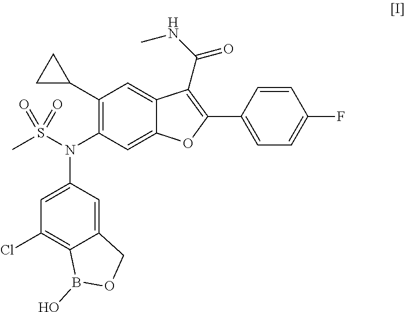 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof,

in combination with one or more additional compounds selected from the group consisting of Telaprevir (Incivek®), Boceprevir (Victrelis®), ABT-450, Faldaprevir (BI-201335), Asunaprevir (BMS-650032), GS-9256, GS-9857, ABT-493, Vedroprevir (GS-9451), Danoprevir (ITMN-191, RG7227), (Grazoprevir) MK-5172, Vaniprevir (MK-7009), Sovaprevir (ACH-1625), Deldeprevir (Neceprevir) (ACH-2684), Narlaprevir (SCH 900518), Simeprevir (TMC 435), ABT-267, ABT-530, Daclatasvir, Velpatasvir, Ledipasvir, ACH-2928, odalasvir (ACH-3102), PPI-668, AZD-7295, Elbasvir (MK-8742), MK-8408, BMS-986094, MK-3862 (IDX-21437), Sofosbuvir, AL-335, GS-0938, Mericitabine, BCX-5191, IDX-184, ALS-2200 (VX-135), ALS-2158, TMC649128, VX-222, ABT-072, ABT-333, Deleobuvir (BI-207127), Tegobuvir (GS-9190), Setrobuvir (ANA-598), CC-31244, Filibuvir (PF-868554), VCH-916, VCH-759, BMS-791325, TMC-647055, TKM-HCV, or a pharmaceutically salt thereof.

In other embodiments, there is provided a LAP pharmaceutical composition, comprising: the compound of Formula I

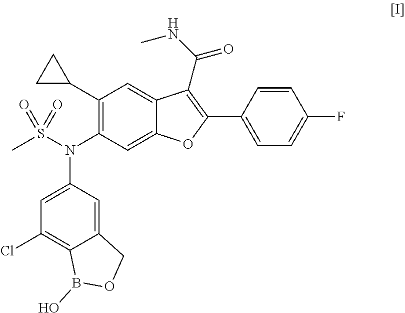 [SMILES: CNC(=O)c1c(-c2ccc(F)cc2)oc2cc(N(c3cc(Cl)c4c(c3)COB4O)S(C)(=O)=O)c(C3CC3)cc12]

or a pharmaceutically acceptable salt thereof,

in combination with any boosting agent, such as, ritonavir. The boosting agent could be dosed simultaneously as the compound of Formula I in the same IV or SC syringe, or it could be dosed separately as an oral tablet or capsule.

Methods for the preparation of the benzofuran derivatives, including the compounds of formula (I) are described in WO2013028371 deriving from U.S. Provisional Application 61/525,440, filed Aug. 19, 2011, which is incorporated herein by reference in its entirety.

The pharmaceutical compositions of the invention are presented as pharmaceutical compositions suitable for parenteral administration. The compositions may also include a safe and effective amount of other active ingredients, such as antimicrobial agents, antiviral agents, or preservatives.

It will be appreciated by those skilled in the art that the amount of active ingredients required for use in treatment will vary according to a variety of factors, including the nature of the condition being treated and the age and condition of the patient, and will ultimately be at the discretion of the attending physician, veterinarian or health care practitioner.

Compositions of the present invention enable patients greater freedom from multiple dosage regimens and ease the needed diligence required in remembering complex daily dosing times and schedules. The compositions of the present invention are particularly suitable for administration as a single dose, monthly, bi-monthly or tri-monthly, or at any interval between 30 and 365 days, including every six or twelve months.

Advantageously, the compositions of the present invention may be administered once.

The compositions of the present invention may be used in combination with other pharmaceutical formulations as a component of a multiple drug treatment regimen. Such combinations could be administered to a subject in one dosage unit, such as a fixed dose combination or it could be administered in separate dosage units.

Compositions of the present invention may also be packaged as articles of manufacture comprising a therapeutically effective amount of a compound of formula (I), or a pharmaceutically acceptable salt thereof; and therapeutically effective amount of one or more of the following: nucleoside NS5B polymerase inhibitors, non-nucleoside NS5B polymerase inhibitors, NS3/4A protease inhibitor, NSSA inhibitor and NS3 protease inhibitor. In one embodiment, the compositions of the present invention could be administered to a subject in combination with one or more of the following HCV treatment compounds: in combination with one or more additional compounds selected from the group consisting of Telaprevir (Incivek®), Boceprevir (Victrelis®), ABT-450, Faldaprevir (BI-201335), Asunaprevir (BMS-650032), GS-9256, GS-9857, ABT-493, Vedroprevir (GS-9451), Danoprevir (ITMN-191, RG7227), (Grazoprevir) MK-5172, Vaniprevir (MK-7009), Sovaprevir (ACH-1625), Deldeprevir (Neceprevir) (ACH-2684), Narlaprevir (SCH 900518), Simeprevir (TMC 435), ABT-267, ABT-530, Daclatasvir, Velpatasvir, Ledipasvir, ACH-2928, odalasvir (ACH-3102), PPI-668, AZD-7295, Elbasvir (MK-8742), MK-8408, BMS-986094, MK-3862 (IDX-21437), Sofosbuvir, AL-335, GS-0938, Mericitabine, BCX-5191, IDX-184, ALS-2200 (VX-135), ALS-2158, TMC649128, VX-222, ABT-072, ABT-333, Deleobuvir (BI-207127), Tegobuvir (GS-9190), Setrobuvir (ANA-598), CC-31244, Filibuvir (PF-868554), VCH-916, VCH-759, BMS-791325, TMC-647055, TKM-HCV, or a pharmaceutically salt thereof.

The packaging material may also have labeling and information related to the pharmaceutical composition printed thereon. Additionally, an article of manufacture may contain a brochure, report, notice, pamphlet, or leaflet containing product information. This form of pharmaceutical information is referred to in the pharmaceutical industry as a “package insert.” A package insert may be attached to or included with a pharmaceutical article of manufacture. The package insert and any article of manufacture labeling provides information relating to the pharmaceutical composition. The information and labeling provides various forms of information utilized by health-care professionals and patients, describing the composition, its dosage and various other parameters required by regulatory agencies such as the United States Food and Drug Agencies.

The present invention further provides the following embodiments:
(a) A parenteral pharmaceutical composition comprising an effective amount of compound of formula (I) or a pharmaceutically acceptable salt thereof, for the cure of HCV infection, or prevention of HCV infection in an individual at risk of being infected by HCV, wherein the composition is administered intermittently at a time interval of a single treatment;
(b) The composition according to (a) wherein the composition is administered once every two weeks.
(c) The composition according to (a) wherein the composition is administered once every month.
(d) The composition according to any one of (a) to (c) wherein the effective amount of compound of formula (I) or a pharmaceutically acceptable salt thereof is selected such that the blood plasma concentration of compound of formula (I) in a subject is kept during a prolonged period of time at a level between a maximum blood plasma level which is the blood plasma level that causes significant side effects and the minimum blood plasma level that is the lowest blood plasma level that causes a compound of formula (I) to provide effective treatment or prevention of HCV infection;
(e) The composition according to (d) wherein the blood plasma level of a subject is kept at a level equal to or above about 150 ng/ml, in particular equal to or above about 600 ng/ml;
(f) The composition according to any one of (a) to (e), wherein the composition is administered subcutaneously or intramuscularly;
(g) The composition according to any one of (a) to (f), which comprises the aforementioned surfactant system comprising polysorbate and/or polyvinylpyrrolidone;
(h) A method for the treatment or prevention or cure of an HCV infection in a human comprising administering to the human a pharmaceutical composition according to any of the above (a) to (g).

The dose of a compound of formula (I) administered, which is the amount of the compound of formula (I) in the parenteral composition for use in the invention, may be selected such that the blood plasma concentration of the compound of formula (I) in a subject is kept during a prolonged period of time above a minimum blood plasma level. The term “minimum blood plasma level” (or Cₘᵢₙ) in this context refers to the lowest efficacious blood plasma level, that is, the blood plasma level of the compound of formula (I) that provides effective prevention or treatment HCV infection. In the case of transmission of HCV from an individual infected by HCV to an individual not infected by HCV, this is the lowest blood plasma level that is effective in inhibiting said transmission.

The blood plasma level of the compound of formula (I) in a subject may be kept at a level above a minimum blood plasma level of about 170 ng/ml, about 700 ng/ml, or about 1000 ng/ml. The blood plasma levels of the compound of formula (I) in a subject may be kept above these minimum blood plasma levels because at lower levels the drug may no longer be effective, thereby increasing the risk of transmission of HCV infection, and may be suboptimal for treatment of HCV infected subjects. Plasma levels of the compound of formula (I) may be kept at higher levels to avoid the development of HCV mutations, while maintaining a safety margin.

An advantage of the mode of administration of the compound of formula (I) is that high Cₘᵢₙ levels can be achieved without a commensurate high Cₘₐₓ, which could mitigate potential side effects associated with Cₘₐₓ.

The effective amount of compound (I) to be administered may be selected such that the blood plasma concentrations in a subject (or patient) are kept during a prolonged period of time at a level between a maximum plasma level (or Cₘₐₓ) and the minimum blood plasma level (or Cₘᵢₙ).

In some embodiments the blood plasma level of compound (I) in a subject may be kept between the minimum blood plasma level (or Cₘᵢₙ as specified above) and the lower maximum plasma level of compound (I) (or Cₘₐₓ) which is defined as the level that corresponds to the lowest blood plasma level where compound (I) acts therapeutically. The lowest level where compound (I) acts therapeutically is the lowest blood plasma level that is effective in inhibiting replication of HCV in individuals infected by HCV so that the viral load of HCV is relatively low, for example where the viral load (represented as the number of copies of viral RNA in a specified volume of serum) is below about 200 copies/ml, in particular below about 100 copies/ml, more particularly below 50 copies/ml, specifically below the detection limit of the assay for HCV.

As mentioned above, the blood plasma levels of compound (I) depend on the amount of active ingredient in each parenteral dosage administered. However, it also depends on the frequency of the administrations (i.e. the time interval between each administration). Both parameters can be used to direct the blood plasma levels to the desired values. The dose may be higher where administrations are less frequent or a single treatment represents the course of therapy.

Although the plasma levels of compound (I) should remain below a maximum or above a minimum value, they may surpass the maximal value or drop below the minimal value during relatively short periods of time, which is usually kept as short as possible. The maximum and minimum plasma levels therefore can be expressed as mean plasma levels during a certain period of time.

In some instances there may be a small initial plasma concentration peak shortly after administration, after which the plasma levels achieve a steady-state.

The compositions of the present invention conveniently allow administration of the compound of Formula I in unit dosage form containing, for example, from about 1 mg to about 1000 mg, from about 20 mg to about 100 mg, from about 20 mg to about 300 mg, from about 25 mg to about 800 mg, from about 25 mg to about 100 mg, from about 100 mg to about 200 mg, from about 200 mg to about 400 mg, from about 100 mg to about 800 mg, from about 100 mg to about 600 mg, from about 100 mg to about 400 mg per unit dosage form, or from about 400 mg to about 800 mg. In one embodiment, the unit dose is from about 400 mg to about 800 mg, which is administered to the subject once. In another embodiment, the subject could be dosed once with 800 mg which may be split into multiple sequential injections.

The unit dose concentration of the compound of Formula I in the formulation may be selected from any of the following ranges: 5-25 mg/mL, 25-50 mg/mL, 50-150 mg/mL, or 150-300 mg/mL.

Once administered, the blood plasma levels of compound (I) in a subject may be more or less stable. After initial rise of the blood plasma levels, a steady state mode may be achieved during a prolonged period of time. By “steady state” is meant the condition in which the amount of drug present in the blood plasma of a subject stays at more or less the same level over a prolonged period of time. The plasma levels of compound (I) may then gradually decrease over time, and when the minimum plasma level is reached, then the next dose of compound (I) may be administered. Alternatively, the virus may be cleared through a single treatment intervention. The term “stays at more or less the same level” does not exclude that there can be small fluctuations of the plasma concentrations within an acceptable range, for example, within about 30%, about 20%, or about 10%.

The parenteral compositions of compound (I) may be administered by intravenous injection or, preferably by subcutaneous or intramuscular administration.

The present invention is based on the use of parenteral compositions of the active ingredient compound (I) and therefore the nature of the carrier is selected for suitability for parenteral administration. The carrier in most cases will comprise sterile water, in although other ingredients, for example, to aid solubility, may be included. Injectable solutions or suspensions, for example, may be prepared in which the carrier comprises saline solution, glucose solution or a mixture of saline and glucose solution. Further, the carrier may contain the surfactant system mentioned above such as polysorbate and Poloxamers.

The parenteral pharmaceutical composition comprising compound (I) of the present invention is long-acting. Accordingly, the composition is useful for the treatment or prevention of HCV infection with administration at long time intervals, compared with conventional compositions or with other compounds similar to compound (I) in chemical structure. The compositions of the present invention can be administered to a patient once or intermittently, e.g., once per week, once per month, once per every 2 months, or one per every 3 months.

Therefore, the compositions of the present invention and an administration by subcutaneous (SC) or intramuscular (IM) injection using the same can lead to a remarkable reduction or elimination of medication (pill) burden and difficulty in patient compliance. Further, such intermittent administration of a composition of the present invention can contribute to maintaining therapy at appropriate compliance which leads to prevention of emergence of drug resistant HCV while the virus is cleared.

In embodiment, the compound of Formula I formulation is a liquid suspension form for a bolus intramuscular or subcutaneous administration at a concentration that ranges from 10 mg/ml to 250 mg/ml and having an injection volume of up to 4 ml (e.g., 2 injections, each 2 ml).

### EXAMPLES

The following examples further describe and exemplify particular embodiments within the scope of the present Invention. The examples are given solely for illustration and are not to be construed as limitations as many variations are possible without departing from spirit and scope of the Invention.

The compound of Formula I, may be synthesized by one of skill in the art by following the teachings of PCT Published Application No. WO2013028371 deriving from U.S. Provisional Application 61/525,440, filed Aug. 19, 2011 which disclose a class of compounds useful in the treatment of HCV infection.

A Thermo Orion 9110DJWP microelectrode and a Metrohmn 827 pH Meter were used for pH measurements. An Advanced Micro-Osmometer 3320 was used for osmolarity measurements. A Retsch PM400 planetary mill was used for wet bead milling.

### Example 1: Preparation of LAP Vehicle

1.0 g of Polysorbate 80 was added to a 0.5 L volumetric flask. About 100 mL of Water for Injection (WFI) was added to the flask to dissolve. 8.5 g of Plasdone K29/32 was added to the flask with an additional 300 mL of WFI. The contents were stirred with a stir bar to dissolve. Phosphate buffer: 0.11039 g NaH₂PO₄; 0.27598 g NaH₂PO₄:H₂O; and 0.22572 g Na₂HPO₄ along with 4.16389 g NaCl as isotonicity agent was added. The mixture was again stirred to dissolve and then was q.s. to 500 mL. The solution was filtered through a 0.22 micrometer Corning filter. The resultant LAP vehicle was 1.7% w/v Plasdone K29/32 and 0.2% w/v Polysorbate 80 in phosphate buffer: 0.004M NaH₂PO₄ and 0.006M Na₂HPO₄.

### Example 2: Homogenized Suspension Compositions

(a) 2.5 mg/ml Homogenized Solution of the Compound of Formula I in LAP Vehicle for Subcutaneous Injection (SC).

17.5 mg of the compound of Formula (I) was added to a clear 10 ml sterile vial with a crimp cap. The LAP Vehicle (as prepared in Example 1) was added to a weight of 7 grams. The solution was homogenized using a handheld Polytron PT1200F homogenizer for 1-2 minutes with a speed increasing from low to near max. The solution was then stirred at ambient room temperature. The resulting title solution had an osmolarity of 313 mOsm/kg and pH of 5.49. The solution was utilized for 5 mg/kg SC injections.

(b) 10.0 mg/ml Homogenized Solution of the Compound of Formula I in LAP Vehicle for SC and IM (Intra-Muscular) Injection

40 mg of the compound of Formula (I) was added to a clear 10 ml sterile vial with a crimp cap. The LAP Vehicle (as prepared in Example 1) was added to a weight of 4 grams. The solution was homogenized using a handheld Polytron PT1200F homogenizer for 1-2 minutes with a speed increasing from low to near max. The solution was then stirred at ambient room temperature. The resulting title solution had an osmolarity of 330 mOsm/kg and pH of 5.47. The solution was utilized for 5 mg/kg IM injections.

### Example 3: Wet Bead Milling Formulations

(a) Preparation of Wet Bead Milled Stock Suspension of the Compound of Formula I in LAP Vehicle

1000 mg of the compound of Formula I is weighed into a 50 mL milling vessel. compound of Formula I was added to a clear 10 ml sterile vial with a crimp cap. The LAP Vehicle (as prepared in Example 1) was added to a weight of 10 grams thereby yielding a 100 mg/ml suspension. Beads were added at 4× suspension volume and the milling vessel was sealed with security tape. Milling was started at 250 rpm for 2 hours using a planetary mill PM400 with a 15 minute interval. After 2 hours the milling vessel was left in the planetary mill for 1.5 hours at ambient room temperature. The beads were filtered using a 25 mm Easy pressure Syringe Filter Holder (screen size: 149 micrometers). A milky suspension was collected and stirred with a stir bar to defoam. The resulting wet bead milled (WBM) suspension had an osmolarity of 303 mOsm/kg and pH of 7.2. The solution was utilized for preparing the WBM suspensions following.

(b) 10.0 mg/ml WBM Suspension of the Compound of Formula I in LAP Vehicle for IM Injection

0.294 g of WBM suspension of Example 3(a) was added to a clear 5 ml sterile vial with a crimp cap. The LAP Vehicle (as prepared in Example 1) was added to a weight of 3 grams. The contents were swirled to mix. The resulting title solution had a pH of 5.28. The solution was utilized for 5 mg/kg IM injections.

(c) 2.5 mg/ml WBM Suspension of the Compound of Formula I in LAP Vehicle for SC Injection

0.122 g of WBM suspension of Example 3(a) was added to a clear 5 ml sterile vial with a crimp cap. The LAP Vehicle (as prepared in Example 1) was added to a weight of 5 grams. The contents were swirled to mix. The resulting title solution had a pH of 5.57. The solution was utilized for 5 mg/kg SC injections.

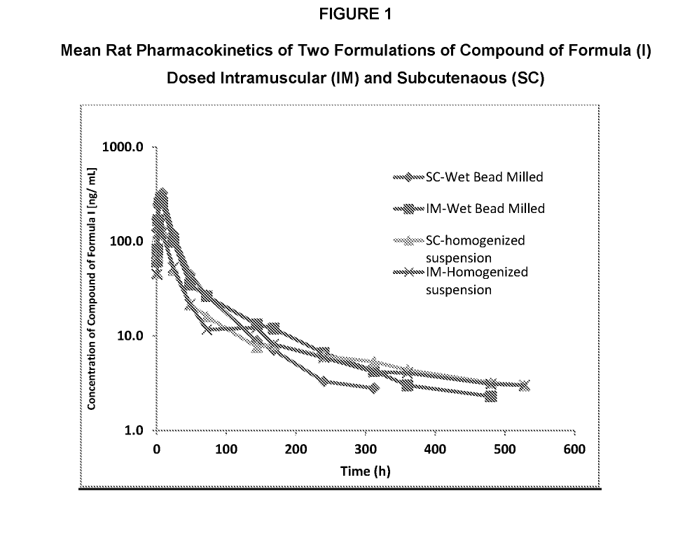

Injections were made in Sprague-Dawley rats SC and IM at 5 mg/kg doses with T_{1/2}, Cₘₐₓ, Tₘₐₓ, and AUC being measured. Results are shown in Table 1 and FIG. 1.

TABLE 1

| Route of | | | | Cₘₐₓ | | |
|---|---|---|---|---|---|---|
| Administration | Formulation | Dose | T_{1/2} (days) | (ng/ml) | Tₘₐₓ (h) | AUCₗₐₛₜ(h*μg/ml) |
| SC | WBM | 5 | 27 | 327.3 ± 58.3 | 6.7 ± 1.2 | 10.36 ± 2.1 |
| | Homogenized | 5 | 12 | 130 ± 17.6 | 6.7 ± 2.3 | 6.4 ± 1.0 |
| | suspension | | | | | |
| IM | WBM | 5 | 5 | 293 ± 110.4 | 5.3 ± 1.2 | 9.95 ± 2.9 |
| | Homogenized | 5 | 8 | 155.7 ± 15.0 | 5.3 ± 1.2 | 6.28 ± 0.74 |
| | suspension | | | | | |

### Example 4: Determination of the Pharmacokinetics of Compound of Formula I in Two Formulations after a Single Intramuscular Administration to Dogs (n=3 Per Group)

Dose Administration:

Individual doses were calculated based on body weights recorded on the day of dose administration. Animals were given an intramuscular (IM) injection. The number of injection sites was based upon dose volume and was recorded in the data. The IM injection sites were monitored and any unusual observations noted throughout the duration of the study and recorded in the raw data.

Sample Collection, Handling, Storage, and Shipment:

Blood was collected into tubes containing K₂EDTA anticoagulant. Blood (approximately 1 mL) was collected from each animal predose and at 0.5, 1, 2, 4, 8, 24, 48, 72, 96, 120, 144, 168, 192, 264, 336, 432, 504, 600, 672, 768, 840, 936, 1008, 1104, 1176, 1272, 1344, 1440, 1512, 1608, and 1680 hours post test article dose. Blood was collected via a jugular vein. Another vein may have been used as an alternative blood collection site and the site recorded in the data.

Sample Handling and Storage:

Blood for pharmacokinetics was maintained on wet ice or at approximately 4° C. prior to centrifugation to obtain plasma. Centrifugation began within 1 hour of collection. Plasma was acidified by mixing with an equal volume of 50 mM (in water) citrate buffer (pH ˜4.0). For each sample, all plasma (up to tube volume) was placed into 96-well plate with individual tubes for each and stored at <−60° C. until shipment. Tubes were arranged by time point by group/row with time points from left to right.

Sample Analysis:

Plasma samples were analyzed for concentrations of the compound of Formula I by bioanalytical services using a liquid chromatography/mass spectrometry (LC-MS/MS) method.

Pharmacokinetic Analysis:

Pharmacokinetic analyses included determination of maximum concentration (Cₘₐₓ), time to maximum concentration (Tₘₐₓ), total area under the curve (AUC), and half-life (t_{1/2}).

TABLE 2

| Component | Function | Concentration (mg/ml) | |
|---|---|---|---|
| Compound of Formula I | Active | 50-250 | |
| Poloxamer 188, Tween 20, | Wetting agent | 20-120 | |
| Tween 80 | | | |
| PEG3350 | Stabilizer | 20 | |
| Mannitol | Tonicity agent | 30-45 | |
| Sodium acetate or sodium | Buffer | 0-20 | mM |
| phosphate | | | |

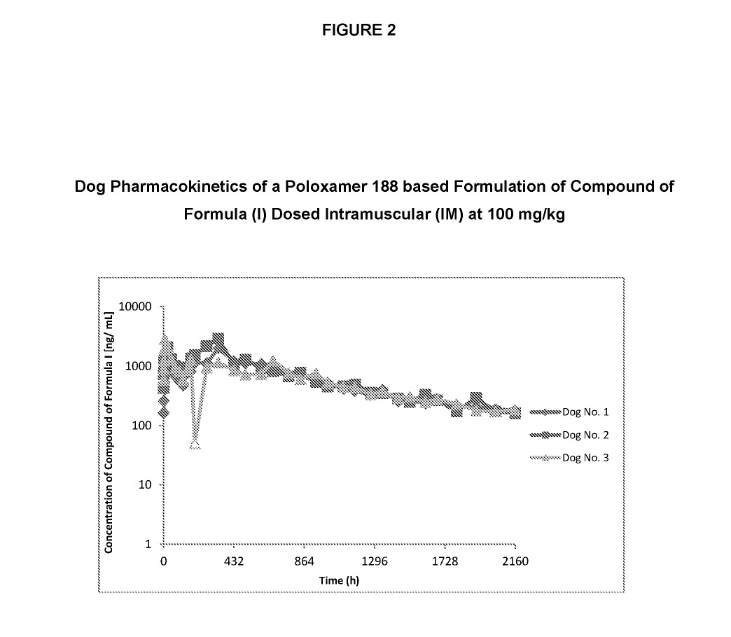

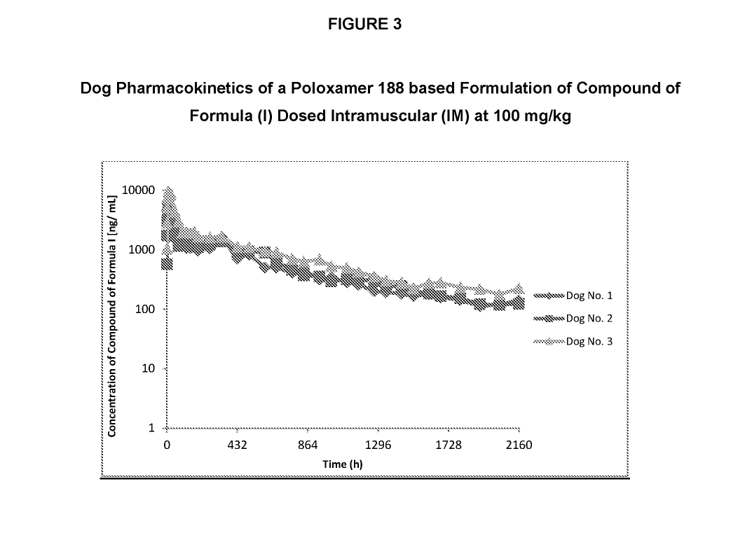

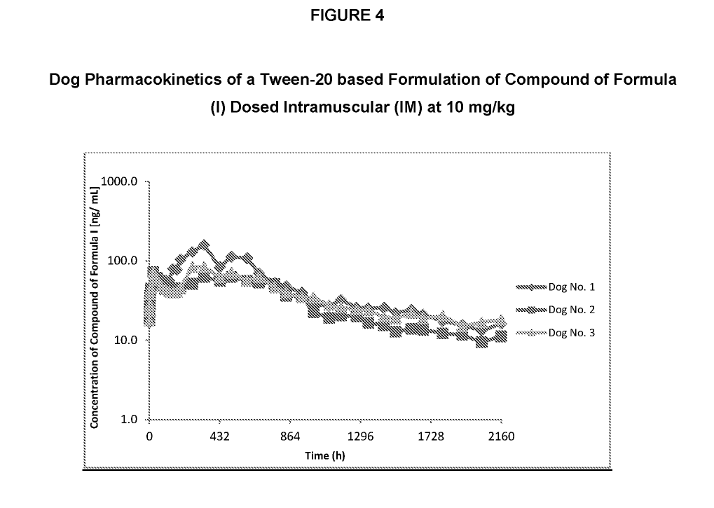

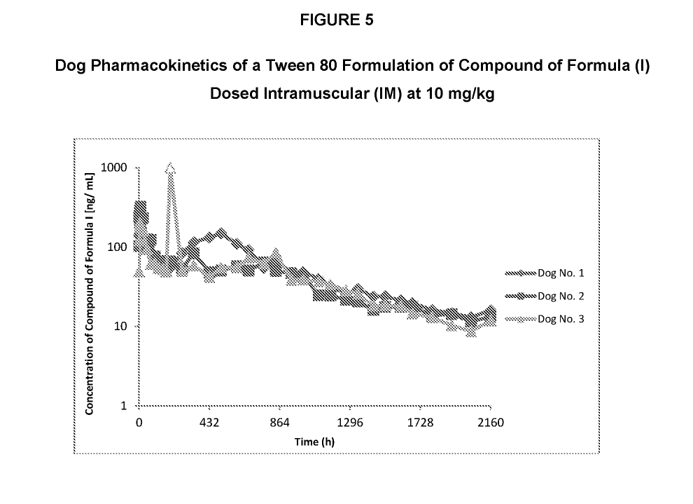

FIG. 2 represents individual concentration-time plots from dogs administered a micronized suspension of compound of formula I formulated with Poloxamer 188 as the wetting agent at a dose level of 100 mg/kg. FIG. 3 represents individual concentration-time plots from dogs administered a nanomilled suspension of compound of formula I formulated with Poloxamer 188 as the wetting agent at a dose level of 100 mg/kg. FIG. 4 represents individual concentration-time plots from dogs administered a micronized suspension of compound of formula I formulated with Tween 20 as the wetting agent at a dose level of 10 mg/kg. FIG. 5 represents individual concentration-time plots from dogs administered a nanomilled suspension of compound of formula I formulated with Tween 80 as the wetting agent at a dose level of 10 mg/kg.

What is claimed is:

1. A long acting parenteral (LAP) pharmaceutical composition comprising a compound of Formula I: 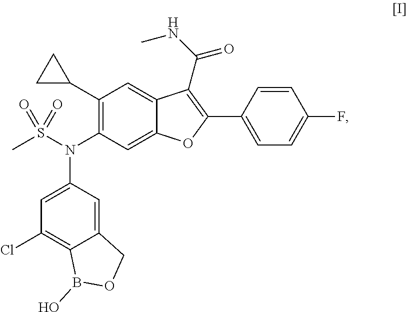 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof.

2. A method for the treatment of an HCV infection in a human having an HCV infection, comprising: administering to the human a LAP pharmaceutical composition including at least one benzofuran derivative or a pharmaceutically acceptable salt thereof.

3. A method for the treatment of an HCV infection in a human having an HCV infection, comprising: administering to the human a LAP pharmaceutical composition comprising a compound of Formula I 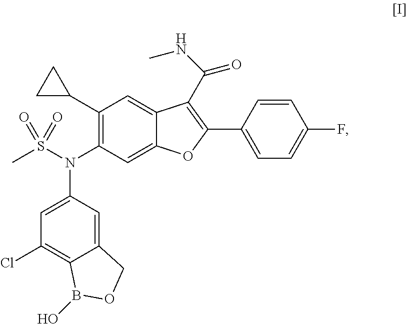 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof.

4. A method for the prevention of an HCV infection in a human having an HCV infection, comprising: administering to the human a LAP pharmaceutical composition including at least one benzofuran derivative or a pharmaceutically acceptable salt thereof.

5. A method for the prevention of an HCV infection in a human having an HCV infection, comprising: administering to the human a LAP pharmaceutical composition including the compound of Formula I 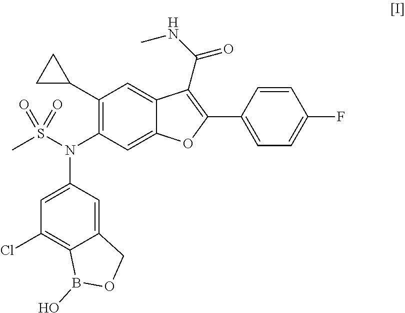 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof.

6. A method for curing an HCV infection in a human having an HCV infection, comprising: administering to the human a LAP pharmaceutical composition comprising the compound of Formula I 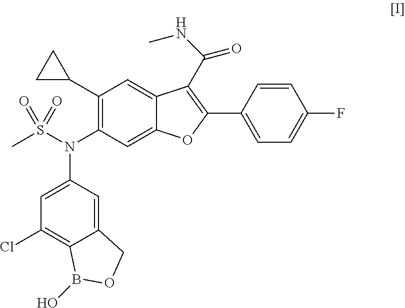 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof.

7. The pharmaceutical composition according to claim 1, further comprising a surfactant system in an amount ranging from about 0.1% (w/v) to about 10% (w/v) surfactant, in an amount ranging from about 1% (w/v) to about 8% (w/v) surfactant, or in an amount of about 2% (w/v) surfactant.

8-10. (canceled)

11. The pharmaceutical composition according to claim 7, wherein the surfactant system comprises a surfactant selected from the group consisting of polyoxyethylene sorbitan fatty acid esters, poloxamers, sorbitan esters of fatty acids (SPAN), polyethoxylated castor oil and its derivatives, tocopheryl polyethylene glycol succinate, and polyvinyl alcohols.

12. The pharmaceutical composition according to claim 7, wherein the surfactant system comprises a surfactant that is polysorbate 20 or polysorbate 80.

13. (canceled)

14. The pharmaceutical composition according to claim 7, wherein the surfactant system comprises a stabilizer that is selected from the group consisting of polyethylene glycols, carboxymethylcellulose calcium, carboxymethylcellulose sodium, methylcellulose, hydroxyethylcellulose, hydroxypropylcellulose, hydroxymethylpropylcellulose, polysaccharides, hyarluronic acid, polyvinyl alcohol (PVA) and polyvinylpyrrolidone (PVP).

15. The pharmaceutical composition according to claim 14, wherein the surfactant system comprises a stabilizer that is polyethylene glycolor PEG-3350.

16. (canceled)

17. The pharmaceutical composition according to claim 14, wherein the surfactant system comprises a stabilizer in an amount that ranges from about 1% (w/v) to about 5% (w/v) stabilizer or about 2% (w/v) stabilizer.

18. (canceled)

19. The pharmaceutical composition according to claim 7, wherein the surfactant system comprises a buffer salt at a concentration of about 10 mM.

20. The pharmaceutical composition according to claim 19, wherein the surfactant system comprises a buffer salt that is acetate buffered saline.

21. (canceled)

22. The pharmaceutical composition according to claim 1, wherein the compound of Formula I is in a crystalline form.

23. The pharmaceutical composition according to claim 22, wherein the compound of Formula I is in a crystalline microparticle form.

24. The pharmaceutical composition according to claim 23, wherein the compound of Formula I is in a crystalline microparticle form and wherein the crystalline microparticles of the compound of Formula I range in size from about 0.05 μm to about 100 μm.

25. The pharmaceutical composition according to claim 23, wherein the compound of Formula I is in the crystalline form prior to encapsulating into a microparticle and combining with a surfactant system.

26. The pharmaceutical composition according to claim 25, wherein the compound of Formula I is encapsulated in a polymer.

27. The pharmaceutical composition according to claim 26, wherein the compound of Formula I is encapsulated in a polymer that comprises poly (lactic-co-glycolic) acid.

28. The method according to claim 2, wherein the human is administered the LAP pharmaceutical composition comprising the compound of Formula I, on a dosing regimen ranging from about every week to about every three months, on a dosing regimen ranging from about every week to about every two months, on a dosing regimen that is monthly, on a dosing regimen that is only one to two administrations or on a dosing regimen that is only one administration.

29-32. (canceled)

33. The method according to claim 28, wherein the administration comprises an injection.

34. (canceled)

35. A LAP pharmaceutical composition, comprising: a compound of Formula I 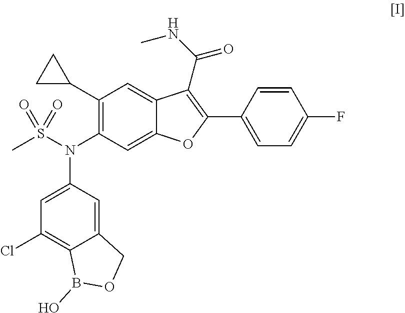 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof, in combination with one or more additional compounds selected from the group consisting of Telaprevir (Incivek®), Boceprevir (Victrelis®), ABT-450, Faldaprevir (BI-201335), Asunaprevir (BMS-650032), GS-9256, GS-9857, ABT-493, Vedroprevir (GS-9451), Danoprevir (ITMN-191, RG7227), (Grazoprevir) MK-5172, Vaniprevir (MK-7009), Sovaprevir (ACH-1625), Deldeprevir (Neceprevir) (ACH-2684), Narlaprevir (SCH 900518), Simeprevir (TMC 435), ABT-267, ABT-530, Daclatasvir, Velpatasvir, Ledipasvir, ACH-2928, odalasvir (ACH-3102), PPI-668, AZD-7295, Elbasvir (MK-8742), MK-8408, BMS-986094, MK-3862 (IDX-21437), Sofosbuvir, AL-335, GS-0938, Mericitabine, BCX-5191, IDX-184, ALS-2200 (VX-135), ALS-2158, TMC649128, VX-222, ABT-072, ABT-333, Deleobuvir (BI-207127), Tegobuvir (GS-9190), Setrobuvir (ANA-598), CC-31244, Filibuvir (PF-868554), VCH-916, VCH-759, BMS-791325, TMC-647055, TKM-HCV, or a pharmaceutically salt thereof.

36. A method for the treatment of an HCV infection in a human having an HCV infection, comprising: administering to the human a LAP pharmaceutical composition including the compound of Formula I 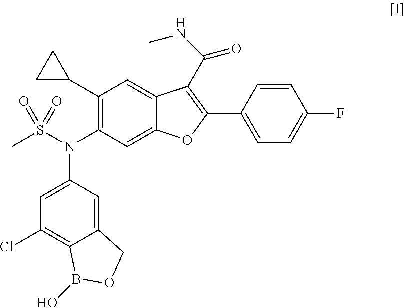 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof, in combination with one or more additional compounds selected from the group consisting of Telaprevir (Incivek®), Boceprevir (Victrelis®), ABT-450, Faldaprevir (BI-201335), Asunaprevir (BMS-650032), GS-9256, GS-9857, ABT-493, Vedroprevir (GS-9451), Danoprevir (ITMN-191, RG7227), (Grazoprevir) MK-5172, Vaniprevir (MK-7009), Sovaprevir (ACH-1625), Deldeprevir (Neceprevir) (ACH-2684), Narlaprevir (SCH 900518), Simeprevir (TMC 435), ABT-267, ABT-530, Daclatasvir, Velpatasvir, Ledipasvir, ACH-2928, odalasvir (ACH-3102), PPI-668, AZD-7295, Elbasvir (MK-8742), MK-8408, BMS-986094, MK-3862 (IDX-21437), Sofosbuvir, AL-335, GS-0938, Mericitabine, BCX-5191, IDX-184, ALS-2200 (VX-135), ALS-2158, TMC649128, VX-222, ABT-072, ABT-333, Deleobuvir (BI-207127), Tegobuvir (GS-9190), Setrobuvir (ANA-598), CC-31244, Filibuvir (PF-868554), VCH-916, VCH-759, BMS-791325, TMC-647055, TKM-HCV, or a pharmaceutically salt thereof.

37. The pharmaceutical composition according to claim 23, wherein the compound of Formula I is in a crystalline microparticle form and wherein the crystalline microparticles of the compound of Formula I range in size from about 0.05 μm to about 100 μm and wherein said crystalline microparticles comprise substantially the same size.

38. The pharmaceutical composition according to claim 23, wherein the compound of Formula I is in a crystalline microparticle form, wherein the crystalline microparticles of the compound of Formula I range in size from about 0.05 μm to about 100 μm, wherein said crystalline microparticles comprise two or more substantially different particle sizes that provide for earlier and later release after administration to a subject and result in varying absorption kinetics.

39. The pharmaceutical composition according to claim 1, wherein the compound of Formula I is in a microparticle form, wherein the microparticles of the compound of Formula I range in size from about 25 μm to about 100 μm, from about 5 μm to about 25 μm, from about 0.5 μm to about 5 μm, from about 0.1 μm to about 5 μm, or from about 0.05 μm to about 0.5 μm.

40-42. (canceled)

43. The pharmaceutical composition according to claim 1, wherein the compound of Formula I is present in an amount ranging from about 20 mg to about 100 mg, from about 100 and to about 200 mg, from about 200 mg to about 400 mg or from about 400 mg to about 800 mg.

44-46. (canceled)

47. The method according to claim 2, wherein the compound of Formula I is administered initially to the subject as a loading dose in amount that ranges from 400 mg to 800 mg and then is administered as a maintenance dose thereafter in an amount that ranges from about 20 mg to about 300 mg.

48. A long acting parenteral (LAP) pharmaceutical composition comprising a compound of Formula I: 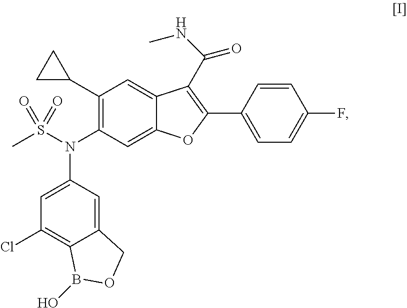 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof, and one or more pharmaceutically acceptable excipients that comprise:
a surfactant;
a stabilizer;
a tonicity agent;
a buffer; and
a solvent.

49. The pharmaceutical composition according to claim 48, wherein the surfactant is Tween 20 or Tween 80.

50. (canceled)

51. The pharmaceutical composition according to claim 49, wherein the buffer is an acetate buffer.

52. The pharmaceutical composition according to claim 49, wherein the tonicity agent is mannitol.

53. (canceled)

54. A long acting parenteral (LAP) pharmaceutical composition comprising a compound of Formula I: 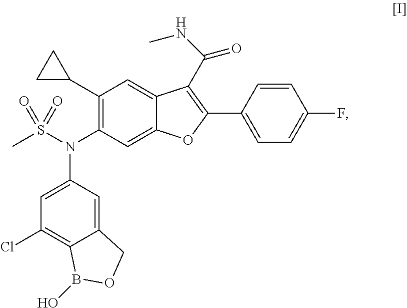 [SMILES: CNC(=O)c1c(-c2ccc(F)cc2)oc2cc(N(c3cc(Cl)c4c(c3)COB4O)S(C)(=O)=O)c(C3CC3)cc12]
or a pharmaceutically acceptable salt thereof, and one or more pharmaceutically acceptable excipients that comprise:
Poloxamer 188;
PEG3350;
D-mannitol,
a buffer comprising sodium acetate or sodium phosphate or both; and
water.

55-59. (canceled)
